(19) United States
(12) Patent Application Publication
(10) Pub. No.: US 2006/0235046 A1
(43) Pub. Date: Oct. 19, 2006

(54) Use of an epidermal growth factor receptor kinase inhibitor (EGFR) in gefitinib resistant patients

(75) Inventors: Charles M. Zacharchuk, Westford, MA (US); Susan E. Quinn, Norwood, MA (US); Patricia Martins, Somerville, MA (US); Lee Greenberger, Montclair, NJ (US); Ante (Bill) Lundberg, Newton, MA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/403,170

(22) Filed: Apr. 12, 2006

Related U.S. Application Data
(60) Provisional application No. 60/671,287, filed on Apr. 14, 2005.

(51) Int. Cl. A61K 31/47

(52) U.S. Cl. 514/313

(57) ABSTRACT

This invention discloses method of treating or inhibiting cancer in a human having at least one of an Exon 19 del E746-A750 and/or an Exon 21 point mutation comprising administering to said human gefitinib and/or iressa alone or in combination with other cytotoxic agents or chemotherapeutic agents and an effective amount of EGFR kinase inhibitor.

This application claims priority from copending provisional application Ser. No. 60/671,287, filed Apr. 14, 2005, the entire disclosure of which is hereby incorporated by reference.

This invention relates to the use of an epidermal growth factor receptor (EGFR) kinase inhibitor in gefitinib resistant patients.

Protein tyrosine kinases are a class of enzymes that catalyze the transfer of a phosphate group from ATP or GTP to tyrosine residue located on protein substrates. Protein tyrosine kinases clearly play a role in normal cell growth. Many of the growth factor receptor proteins function as tyrosine kinases and it is by this process that they effect signaling. The interaction of growth factors with these receptors is a necessary event in normal regulation of cell growth. However, under certain conditions, as a result of either mutation or over expression, these receptors can become deregulated; the result of which is uncontrolled cell proliferation which can lead to tumor growth and ultimately to the disease known as cancer [Wilks A. F., Adv. Cancer Res., 60, 43 (1993) and Parsons, J. T.; Parsons, S. J., Important Advances in Oncology, DeVita V. T. Ed., J.B. Lippincott Co., Phila., 3 (1993)]. Among the growth factor receptor kinases and their proto-oncogenes that have been identified and which are targets of the compounds of this invention are the epidermal growth factor receptor kinase (EGFR kinase, the protein product of the erbB oncogene), and the product produced by the erbB-2 (also referred to as the neu or HER2) oncogene. Since the phosphorylation event is a necessary signal for cell division to occur and since overexpressed or mutated kinases have been associated with cancer, an inhibitor of this event, a protein tyrosine kinase inhibitor, will have therapeutic value for the treatment of cancer and other diseases characterized by uncontrolled or abnormal cell growth. For example, over expression of the receptor kinase product of the erbB-2 oncogene has been associated with human breast and ovarian cancers [Slamon, D. J., et. al., Science, 244, 707 (1989) and Science, 235, 1146 (1987)]. Deregulation of EGF-R kinase has been associated with epidermoid tumors [Reiss, M., et. al., Cancer Res., 51, 6254 (1991)], breast tumors [Macias, A., et. al., Anticancer Res., 7, 459 (1987)], and tumors involving other major organs [Gullick, W. J., Brit. Med. Bull., 47, 87 (1991)]. Because of the importance of the role played by deregulated receptor kinases in the pathogenesis of cancer, many recent studies have dealt with the development of specific PTK inhibitors as potential anti-cancer therapeutic agents [some recent reviews: Burke. T. R., Drugs Future, 17, 119 (1992) and Chang, C. J.; Geahlen, R. L., J. Nat. Prod., 55, 1529 (1992)].

EGFR kinase inhibitors of interest (4-dimethylamino-but-2-enoic acid [4-(3-chloro-4-fluoro-phenylamino)-3-cyano-7-ethoxy-quinolin-6-yl]-amide (EKB-569) and (E)-N-{4-[3-chloro-4-(2-pyridinyl methoxy)aniline]-3-cyano-7-ethoxy-6-quinolinyl}-4-(dimethylamino)-2-butenamide (HKI-272), also a HER-2 inhibitor. While it is important that EKB-569 and HKI-272 work as single anti-cancer agents, it is possible that tyrosine kinase inhibitors may be most effective when given in combination with established chemotherapeutic agents. The studies in this report were designed to determine if EKB-569 or HKI-272 in combination with conventional chemotherapeutic agents (cytotoxic agents) provide better tumor growth inhibition than when either drug is administered alone.

### BRIEF SUMMARY OF THE INVENTION

The present invention relates to a method of treating or inhibiting cancer in a human treated with gefitinib or iressa.

The present invention is a method of treating or inhibiting cancer in a human having at least one of an Exon 19 del E746-A750 and/or an Exon 21 point mutation comprising administering to said human gefitinib alone or in combination with other cytotoxic agents or chemotherapeutic agents and an effective amount of EGFR kinase inhibitor.

The EGFR kinase inhibitor irreversibly inhibits EGFR kinase and has a structure of formula 1: 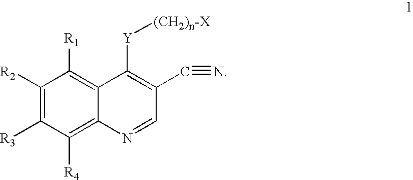
wherein:
X is cycloalkyl of 3 to 7 carbon atoms, which may be optionally substituted with one or more alkyl of 1 to 6 carbon atom groups; or is a pyridinyl, pyrimidinyl, or phenyl ring; wherein the pyridinyl, pyrimidinyl, or phenyl ring may be optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino;
n is 0-1;
Y is —NH—, —O—, —S—, or —NR—;
R is alkyl of 1-6 carbon atoms;
R₁, R₂, R₃, and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzyl, amino, hydroxyamino, alkoxyamino of 1-4 carbon atoms, alkylamino of 1-6 carbon atoms, dialkylamino of 2 to 12 carbon atoms, aminoalkyl of 1-4 carbon atoms, N-alkylaminoalkyl of 2-7 carbon atoms, N,N-dialkylaminoalkyl of 3-14 carbon atoms, phenylamino, benzylamino, 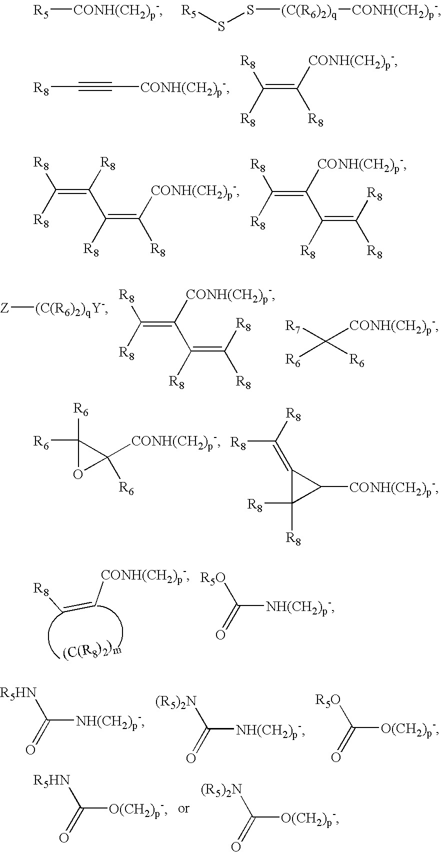
R₅ is alkyl of 1-6 carbon atoms, alkyl optionally substituted with one or more halogen atoms, phenyl, or phenyl optionally substituted with one or more halogen, alkoxy of 1-6 carbon atoms, trifluoromethyl, amino, nitro, cyano, or alkyl of 1-6 carbon atoms groups;
R₆ is hydrogen, alkyl of 1-6 carbon atoms, or alkenyl of 2-6 carbon atoms;
R₇ is chloro or bromo;
R₈ is hydrogen, alkyl of 1-6 carbon atoms, aminoalkyl of 1-6 carbon atoms, N-alkylaminoalkyl of 2-9 carbon atoms, N,N-dialkylaminoalkyl of 3-12 carbon atoms, N-cycloalkylaminoalkyl of 4-12 carbon atoms, N-cycloalkyl-N-alkylaminoalkyl of 5-18 carbon atoms, N,N-dicycloalkylaminoalkyl of 7-18 carbon atoms, morpholino-N-alkyl wherein the alkyl group is 1-6 carbon atoms, piperidino-N-alkyl wherein the alkyl group is 1-6 carbon atoms, N-alkyl-piperidino-N-alkyl wherein either alkyl group is 1-6 carbon atoms, azacycloalkyl-N-alkyl of 3-11 carbon atoms, hydroxyalkyl of 1-6 carbon atoms, alkoxyalkyl of 2-8 carbon atoms, carboxy, carboalkoxy of 1-6 carbon atoms, phenyl, carboalkyl of 2-7 carbon atoms, chloro, fluoro, or bromo;
Z is amino, hydroxy, alkoxy of 1-6 carbon atoms, alkylamino wherein the alkyl moiety is of 1-6 carbon atoms, dialkylamino wherein each of the alkyl moieties is of 1-6 carbon atoms, morpholino, piperazino, N-alkylpiperazino wherein the alkyl moiety is of 1-6 carbon atoms, or pyrrolidino;
m=1-4, q=1-3, and p=0-3;
any of the substituents R₁, R₂, R₃, or R₄ that are located on contiguous carbon atoms can together be the divalent radical —O—C(R8)2—O—;
or a pharmaceutically acceptable salt thereof.

The EGFR kinase inhibitor irreversibly inhibits EGFR kinase and has a structure: 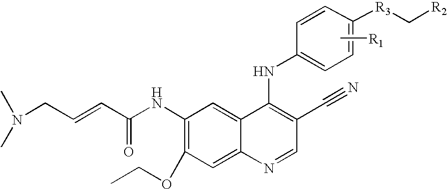
wherein:
R₁ is halogen;
R₂ is a pyridinyl, optionally substituted pyrimidine, thiazole, or an optionally substituted phenyl ring wherein the phenyl or pyrimidine ring may be unsubstituted, mono-substituted, or di-substituted; and
R₃ is —O— or —S—; or a pharmaceutically acceptable salt thereof.

The EGFR kinase inhibitor is (4-dimethylamino-but-2-enoic acid [4-(3-chloro-4-fluoro-phenylamino)-3-cyano-7-ethoxy-quinolin-6-yl]-amide or a pharmaceutically acceptable salt thereof.

The cancer of this invention comprises non-small cell lung cancer.

The following experimental details are set forth to aid in an understanding of the invention, and are not intended, and should not be construed, to limit in any way the invention set forth in the claims that follow thereafter.

### BRIEF DESCRIPTION OF THE DRAWINGS

FIG. 1 contains the EGFR mutation analysis of case 1. Shows the exon 19R deletion (del E746-A750).

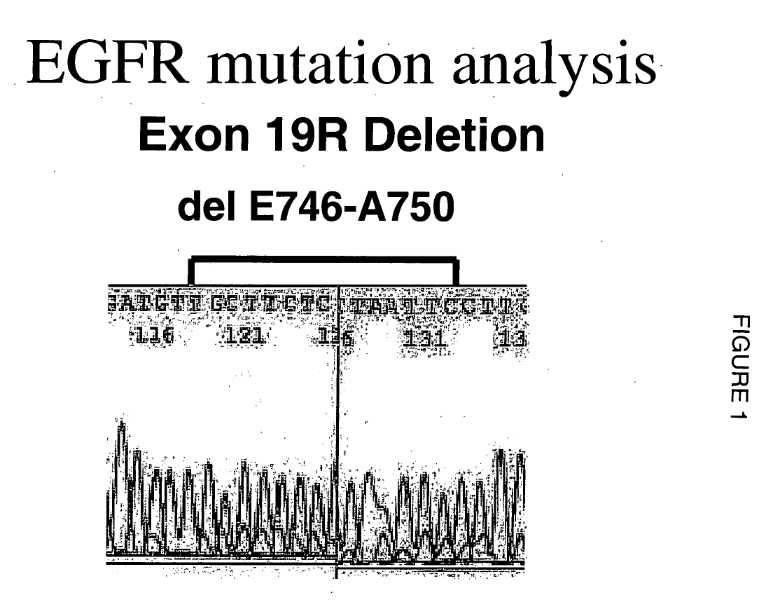

FIG. 2 contains the clinical course for the patient in case 1.

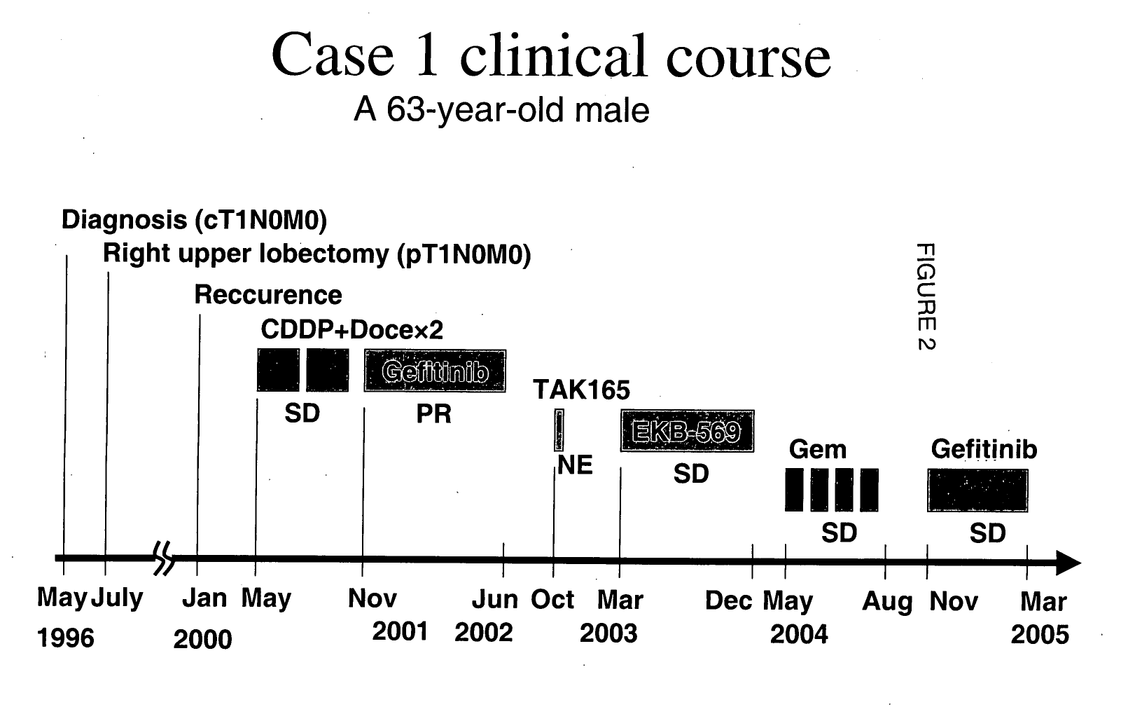

FIG. 3 contains the CT change of the patient in case 1.

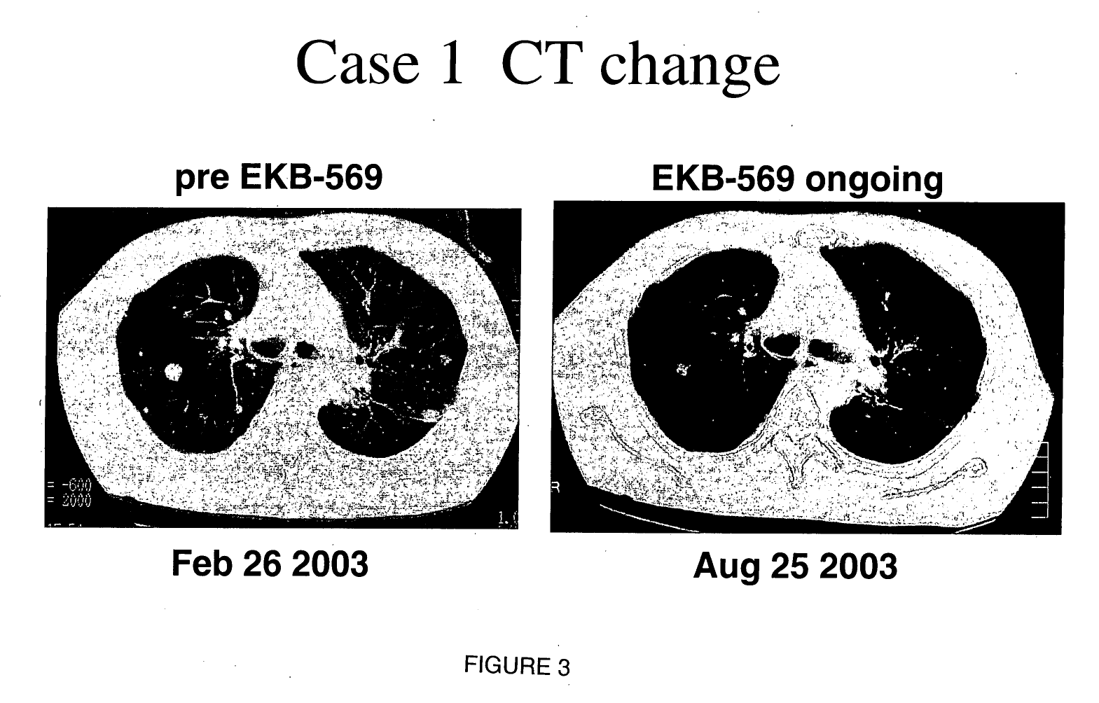

FIG. 4 contains the EGFR mutation analysis of case 2. Shows the exon 21 point mutation (L858R).

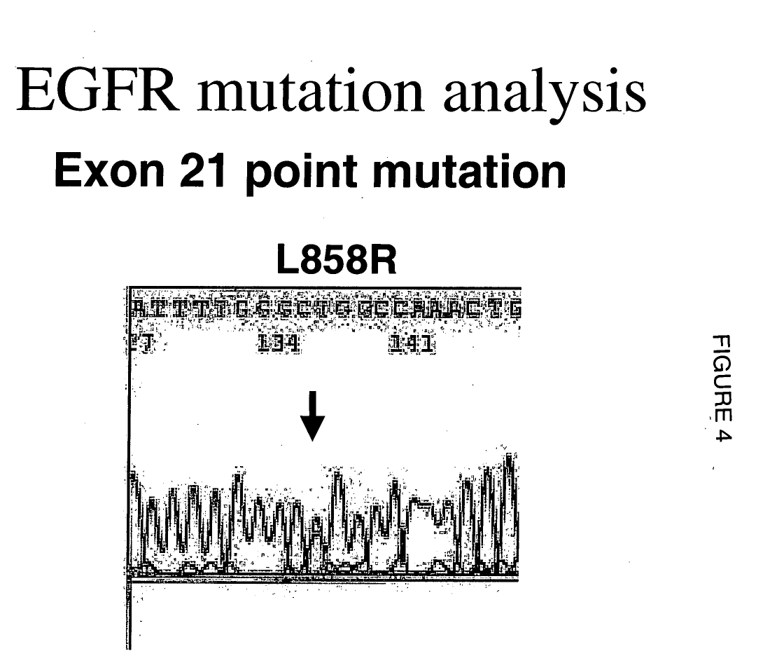

FIG. 5 contains the clinical course for the patient in case 2.

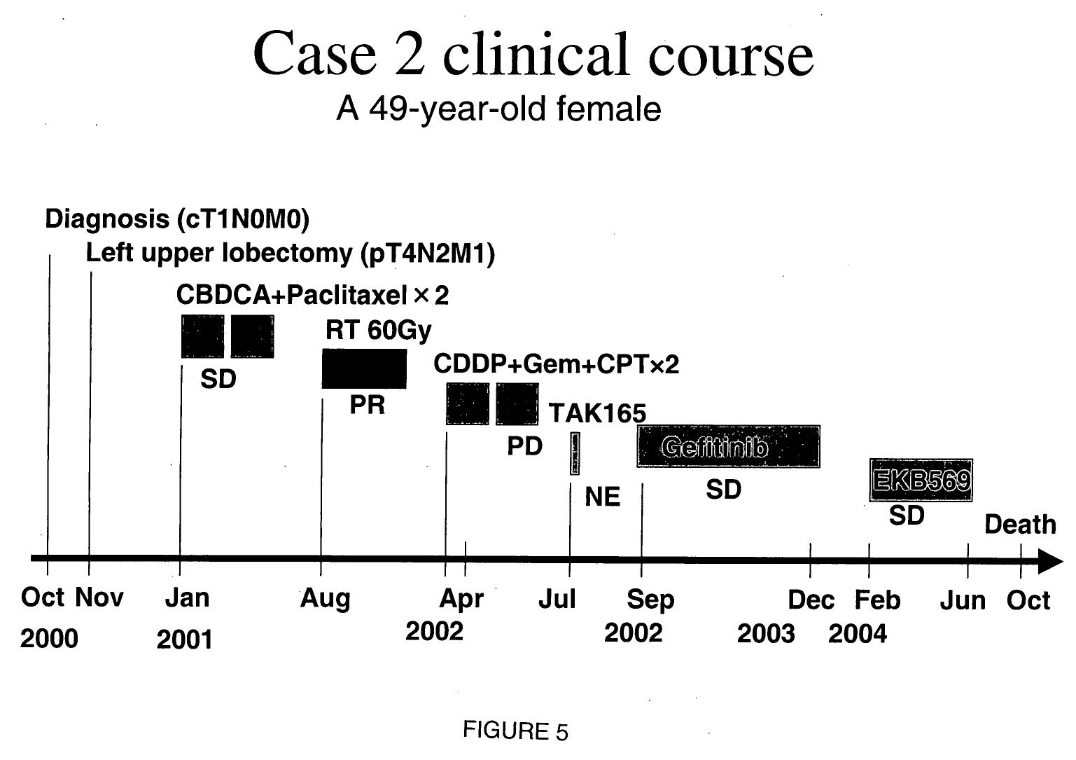

FIG. 6 contains the MRI change for the patient in case 2.

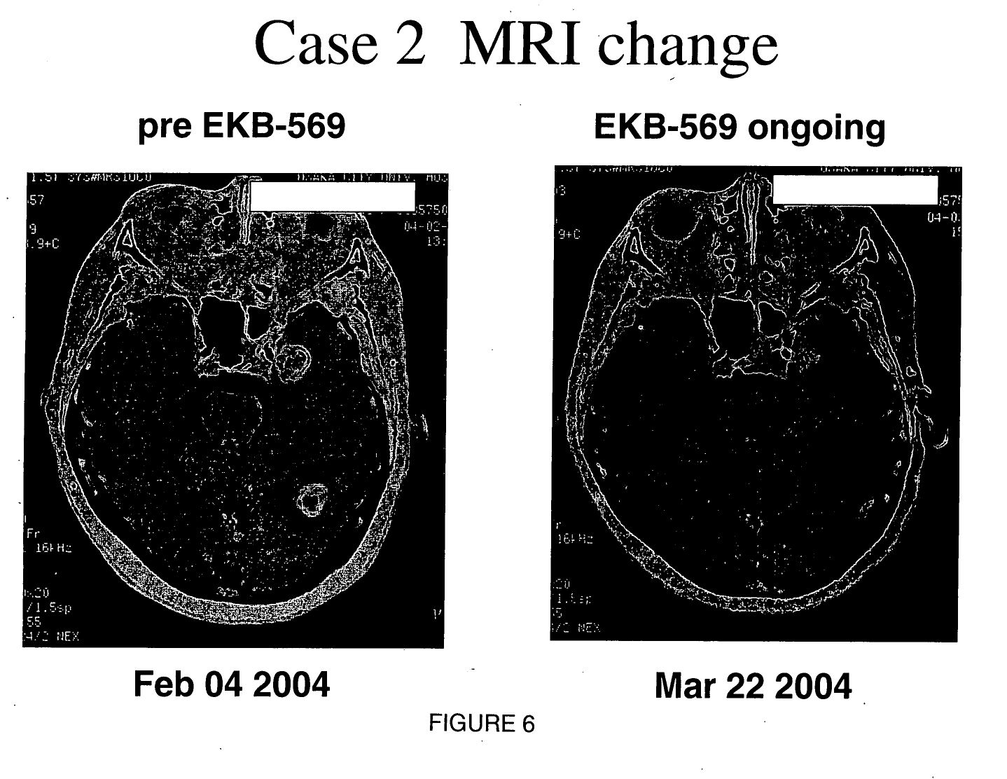

### DETAILED DESCRIPTION OF THE INVENTION

For the purpose of defining the scope of this invention, an EGFR kinase inhibitor is defined as a molecule that inhibits the kinase domain of the EGFR. It is preferred that the EGFR kinase inhibitor irreversibly inhibits EGFR kinase, typically by possessing a reactive moiety (such as a Michael acceptor) that can form a covalent bond with EGFR. The EGFR inhibitor may also have activity as a HER-2 inhibitor.

For purposes of this invention the EGFR kinase inhibitor includes, the following:

Quinazolines of this application, are disclosed in U.S. Pat. Nos. 6,384,051 B1 and 6,288,082, both of which examples are hereby incorporated by reference. These compounds can be prepared according to the methodology described in U.S. Pat. Nos. 6,384,051 B1 and 6,288,082, which is hereby incorporated by reference. The structure of the EGFR kinase inhibitors of Formula 1 are as follows: 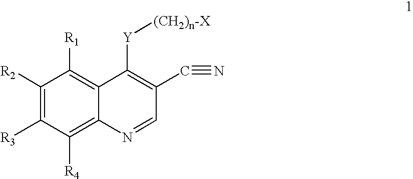
wherein:
X is cycloalkyl of 3 to 7 carbon atoms, which may be optionally substituted with one or more alkyl of 1 to 6 carbon atom groups; or is a pyridinyl, pyrimidinyl, or phenyl ring; wherein the pyridinyl, pyrimidinyl, or phenyl ring may be optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino;
n is 0-1;
Y is —NH—, —O—, —S—, or —NR—;
R is alkyl of 1-6 carbon atoms;
R₁, R₂, R₃, and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzyl, amino, hydroxyamino, alkoxyamino of 1-4 carbon atoms, alkylamino of 1-6 carbon atoms, dialkylamino of 2 to 12 carbon atoms, aminoalkyl of 1-4 carbon atoms, N-alkylaminoalkyl of 2-7 carbon atoms, N,N-dialkylaminoalkyl of 3-14 carbon atoms, phenylamino, benzylamino, 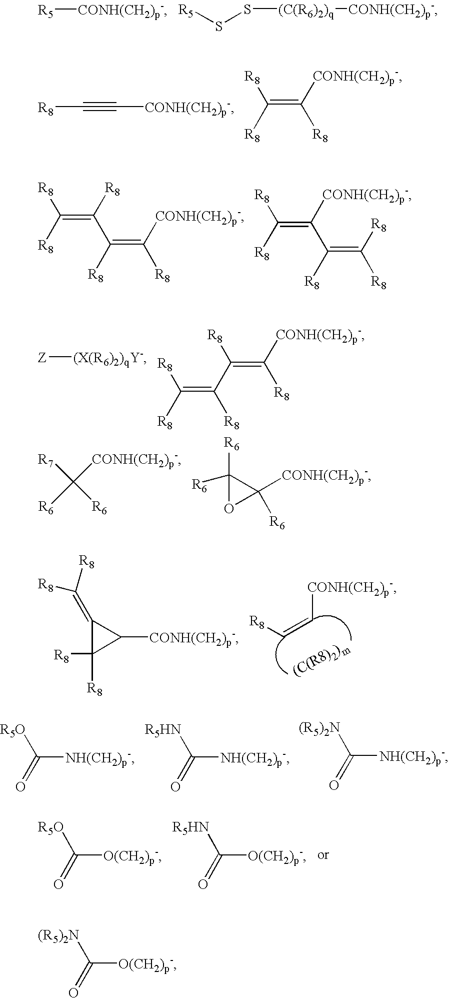
R₅ is alkyl of 1-6 carbon atoms, alkyl optionally substituted with one or more halogen atoms, phenyl, or phenyl optionally substituted with one or more halogen, alkoxy of 1-6 carbon atoms, trifluoromethyl, amino, nitro, cyano, or alkyl of 1-6 carbon atoms groups;
R₆ is hydrogen, alkyl of 1-6 carbon atoms, or alkenyl of 2-6 carbon atoms;
R₇ is chloro or bromo;
R₈ is hydrogen, alkyl of 1-6 carbon atoms, aminoalkyl of 1-6 carbon atoms, N-alkylaminoalkyl of 2-9 carbon atoms, N,N-dialkylaminoalkyl of 3-12 carbon atoms, N-cycloalkylaminoalkyl of 4-12 carbon atoms, N-cycloalkyl-N-alkylaminoalkyl of 5-18 carbon atoms, N,N-dicycloalkylaminoalkyl of 7-18 carbon atoms, morpholino-N-alkyl wherein the alkyl group is 1-6 carbon atoms, piperidino-N-alkyl wherein the alkyl group is 1-6 carbon atoms, N-alkyl-piperidino-N-alkyl wherein either alkyl group is 1-6 carbon atoms, azacycloalkyl-N-alkyl of 3-11 carbon atoms, hydroxyalkyl of 1-6 carbon atoms, alkoxyalkyl of 2-8 carbon atoms, carboxy, carboalkoxy of 1-6 carbon atoms, phenyl, carboalkyl of 2-7 carbon atoms, chloro, fluoro, or bromo;
Z is amino, hydroxy, alkoxy of 1-6 carbon atoms, alkylamino wherein the alkyl moiety is of 1-6 carbon atoms, dialkylamino wherein each of the alkyl moieties is of 1-6 carbon atoms, morpholino, piperazino, N-alkylpiperazino wherein the alkyl moiety is of 1-6 carbon atoms, or pyrrolidino;
m=1-4, q=1-3, and p=0-3;
any of the substituents R₁, R₂, R₃, or R₄ that are located on contiguous carbon atoms can together be the divalent radical —O—C(R₈)₂—O—;
or a pharmaceutically acceptable salt thereof.

The EGFR kinase inhibitor irreversibly inhibits EGFR kinase and has a structure: 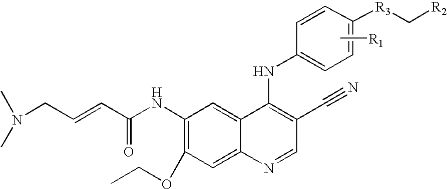
wherein:
R₁ is halogen;
R₂ is a pyridinyl, optionally substituted pyrimidine, thiazole, or an optionally substituted phenyl ring wherein the phenyl or pyrimidine ring may be unsubstituted, mono-substituted, or di-substituted; and
R₃ is —O— or —S—; or a pharmaceutically acceptable salt thereof.

With respect to the quinazolines, the pharmaceutically acceptable salts are those derived from such organic and inorganic acids as: acetic, lactic, citric, tartaric, succinic, maleic, malonic, gluconic, hydrochloric, hydrobromic, phosphoric, nitric, sulfuric, methanesulfonic, and similarly known acceptable acids.

The compounds herein may be administered orally, by intralesional, intraperitoneal, intramuscular or intravenous injection; infusion; liposome-mediated delivery; topical, nasal, anal, vaginal, sublingual, uretheral, transdermal, intrathecal, ocular or otic delivery. In order to obtain consistency in providing the compound of this invention it is preferred that a compound of the invention is in the form of a unit dose. Suitable unit dose forms include tablets, capsules and powders in sachets or vials. Such unit dose forms may contain from 0.1 to 300 mg of a compound of the invention and preferably from 2 to 100 mg. The compounds of the present invention can be administered orally at a dose range of about 0.01 to 100 mg/kg. Such compounds may be administered from 1 to 6 times a day times a day. The effective amount will be known to one of skill in the art; it will also be dependent upon the form of the compound. One of skill in the art could routinely perform empirical activity tests to determine the bioactivity of the compound in bioassays and thus determine what dosage to administer. The compound of the present invention may be delivered locally via a capsule that allows a sustained release of the compound over a period of time. Controlled or sustained release compositions include formulation in lipophilic depots (fatty acids, waxes, oils).

The administration can take the form of dosing a reversible EGFR kinase inhibitor, for example but not limited to gefitinib or iressa alone until resistance is detected during clinical monitoring at which time an effective amount of an irreversible EGFR kinase inhibitor, for example but not limited to EKB or HKI is administered.

The administration can also take the form of dosing a reversible EGFR kinase inhibitor, for example but not limited to gefitinib or iressa alone until resistance is detected during clinical monitoring at which time an effective amount of an irreversible EGFR kinase inhibitor, for example but not limited to EKB or HKI is administered followed by another round of dosing with the reversible EGFR kinase inhibitor as exemplified herein.

The alkyl portion of the alkyl, alkoxy, alkanoyloxy, alkoxymethyl, alkanoyloxymethyl, alkylsulphinyl, alkylsulphonyl, alkylsulfonamido, carboalkoxy, carboalkyl, alkanoylamino aminoalkyl, alkylaminoalkyl, N,N-dicycloalkylaminoalkyl, hydroxyalkyl, and alkoxyalkyl substituents include both straight chain as well as branched carbon chains. The cycloalkyl portions of N-cycloalkyl-N-alkylaminoalkyl and N,N-dicycloalkylaminoalkyl substituents include both simple carbocycles as well as carbocycles containing alkyl substituents. The alkenyl portion of the alkenyl, alkenoyloxymethyl, alkenyloxy, alkenylsulfonamido, substituents include both straight chain as well as branched carbon chains and one or more sites of unsaturation. The alkynyl portion of the alkynyl, alkynoyloxymethyl, alkynylsulfonamido, alkynyloxy, substituents include both straight chain as well as branched carbon chains and one or more sites of unsaturation. Carboxy is defined as a —CO₂H radical. Carboalkoxy of 2-7 carbon atoms is defined as a —CO₂R″ radical, where R″ is an alkyl radical of 1-6 carbon atoms. Carboalkyl is defined as a —COR″ radical, where R″ is an alkyl radical of 1-6 carbon atoms. Alkanoyloxy is defined as a —OCOR″ radical, where R″ is an alkyl radical of 1-6 carbon atoms. Alkanoyloxymethyl is defined as R″CO₂CH₂— radical, where R″ is an alkyl radical of 1-6 carbon atoms. Alkoxymethyl is defined as R″OCH₂— radical, where R″ is an alkyl radical of 1-6 carbon atoms. Alkylsulphinyl is defined as R″SO— radical, where R″ is an alkyl radical of 1-6 carbon atoms. Alkylsulphonyl is defined as R″SO₂— radical, where R″ is an alkyl radical of 1-6 carbon atoms. Alkylsulfonamido, alkenylsulfonamido, alkynylsulfonamido are defined as R″SO₂NH— radical, where R″ is an alkyl radical of 2-6 carbon atoms, an alkenyl radical of 2-6 carbon atoms, or an alkynyl radical of 2-6 carbon atoms, respectively. When X is substituted, it is preferred that it is mono-, di-, or tri-substituted, with monosubstituted being most preferred. It is preferred that of the substituents R₁, R₂, R₃, and R₄, at least one is hydrogen and it is most preferred that two or three be hydrogen. An azacycloalkyl-N-alkyl substituent refers to a monocyclic heterocycle that contains a nitrogen atom on which is substituted a straight or branched chain alkyl radical. A morpholino-N-alkyl substituent is a morpholine ring substituted on the nitrogen atom with a straight or branch chain alkyl radical. A piperidino-N-alkyl substituent is a piperidine ring substituted on one of the nitrogen atoms with a straight or branch chain alkyl radical. A N-alkyl-piperidino-N-alkyl substituent is a piperidine ring substituted on one of the nitrogen atoms with a straight or branched chain alkyl group and on the other nitrogen atom with a straight or branch chain alkyl radical.

The term alkyl includes both straight and branched chain alkyl moieties, preferably of 1-6 carbon atoms. The term alkenyl includes both straight and branched alkenyl moieties of 2-6 carbon atoms containing at least one double bond. Such alkenyl moieties may exist in the E or Z conformations; the compounds of this invention include both conformations. The term alkynyl includes both straight chain and branched alkynyl moieties containing 2-6 carbon atoms containing at least one triple bond. The term cycloalkyl refers to an alicyclic hydrocarbon group having 3-7 carbon atoms.

The term halogen is defined as Cl, Br, F, and 1.

Alkoxy, alkylthio, alkoxyalkyl, alkylthioalkyl, alkoxyalkyloxy and alkylthioalkyloxy are moieties wherein the alkyl chain is 1-6 carbon atoms (straight or branched).

The term alkylamino refers to moieties with one or two alkyl groups wherein the alkyl chain is 1-6 carbons and the groups may be the same or different. The alkyl groups (the same or different) bonded to the nitrogen atom which is attached to an alkyl group of 1-3 carbon atoms.

The compounds herein may contain an asymmetric carbon; in such cases, the compounds of Formula 1 cover the racemate and the individual R and S entantiomers, and in the case were more than one asymmetric carbon exists, the individual diasteromers, their racemates and individual entantiomers.

For purposes of this invention an EGFR kinase inhibitor of interest having a structure of formula 1 includes (4-dimethylamino-but-2-enoic acid [4-(3-chloro-4-fluoro-phenylamino)-3-cyano-7-ethoxy-quinolin-6-yl]-amide) (“EKB-569”).

For purposes of this invention cancer includes non-small cell lung cancer.

Report on 2 Patients with Non-Small Cell Lung Cancer (NSCLC) Who Received EKB-569.

Case 1

A 63 year old male with a smoking history BI 720 (20×38 years) having adenocarcinoma and cT0NOM1 (multiple pulmonary metastasis). Exon 19 deletion E746-A750 is identified. Herception test score was 0+ and EGFR score 2+. The patient was given 25 mg/day EKB-569 for 9 months.

Case 2

A 49 year old female with a smoking history of BI 30 (10×3 years) having adenocarcinoma performance status 1, cT0N3M1 (pulmonary, brain and bone). Exon 21 point mutation L858R. HercepTest score +, EGFR score 3+, EKB-569 35 mg/day for 4 months.

Epidermal growth factor receptor (EGFR) mutations in NSCLC correlate with clinical response and predict prolonged survival after gefitinib (Paez J G, et al, Science, 2004; Lynch T J, et al, N Engl J Med, 2004).

Phase 1 dose-escalation study of EKB-569 was completed in Japanese patients with advanced-stage malignancies known to overexpress EGFR.

EKB-569 was effective in these two patients after treatment with gefitinib and recurrence of NSCLC.

In some cases, re-treatment with gefitinib is effective when NSCLC recurs in patients treated with gefitinib (Kurata T, et al, Ann Oncol, 2004).

Acquired resistance to gefitinib is associated with a second mutation in exon 20 encoding the EGFR kinase domain (Pao W, et al, PLoS Med, 2005)

387,785, a specific and irreversible anilinoquinazoline EGFR inhibitor, strongly inhibited the activity of EGFR encoded by a gene containing a second mutation in the kinase domain (Kobayashi S, et al, N Engl J Med, 2005)

EKB-569, another irreversible EGFR inhibitor, may abrogate the resistance mechanism to gefitinib. One patient had Exon 19 del E746-A750, and the other had Exon 21 point mutation.

What is claimed is:

1. A method of treating or inhibiting cancer in a human having at least one of an Exon 19 del E746-A750 and/or an Exon 21 point mutation comprising administering to said human gefitinib or iressa alone or in combination with other cytotoxic agents or chemotherapeutic agents and an effective amount of EGFR kinase inhibitor.

2. The method according to claim 1, wherein the EGFR kinase inhibitor irreversibly inhibits EGFR kinase.

3. The method according to claim 1, wherein the EGFR kinase inhibitor is a compound of formula 1, having the structure: 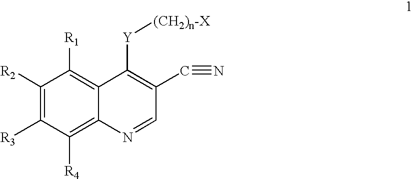
wherein:
X is cycloalkyl of 3 to 7 carbon atoms, which may be optionally substituted with one or more alkyl of 1 to 6 carbon atom groups; or is a pyridinyl, pyrimidinyl, or phenyl ring; wherein the pyridinyl, pyrimidinyl, or phenyl ring may be optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino;
n is 0-1;
Y is —NH—, —O—, —S—, or —NR—;
R is alkyl of 1-6 carbon atoms;
R₁, R₂, R₃, and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzyl, amino, hydroxyamino, alkoxyamino of 1-4 carbon atoms, alkylamino of 1-6 carbon atoms, dialkylamino of 2 to 12 carbon atoms, aminoalkyl of 1-4 carbon atoms, N-alkylaminoalkyl of 2-7 carbon atoms, N,N-dialkylaminoalkyl of 3-14 carbon atoms, phenylamino, benzylamino, 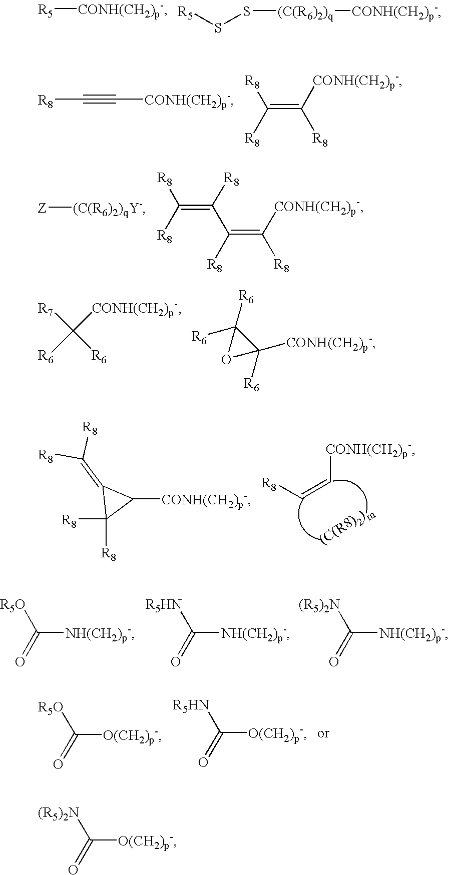
R₅ is alkyl of 1-6 carbon atoms, alkyl optionally substituted with one or more halogen atoms, phenyl, or phenyl optionally substituted with one or more halogen, alkoxy of 1-6 carbon atoms, trifluoromethyl, amino, nitro, cyano, or alkyl of 1-6 carbon atoms groups;
R₆ is hydrogen, alkyl of 1-6 carbon atoms, or alkenyl of 2-6 carbon atoms;
R₇ is chloro or bromo;
R₈ is hydrogen, alkyl of 1-6 carbon atoms, aminoalkyl of 1-6 cabon atoms, N-alkylaminoalkyl of 2-9 carbon atoms, N,N-dialkylaminoalkyl of 3-12 carbon atoms, N-cycloalkylaminoalkyl of 4-12 carbon atoms, N-cycloalkyl-N-alkylaminoalkyl of 5-18 carbon atoms, N,N-dicycloalkylaminoalkyl of 7-18 carbon atoms, morpholino-N-alkyl wherein the alkyl group is 1-6 carbon atoms, piperidino-N-alkyl wherein the alkyl group is 1-6 carbon atoms, N-alkyl-piperidino-N-alkyl wherein either alkyl group is 1-6 carbon atoms, azacycloalkyl-N-alkyl of 3-11 carbon atoms, hydroxyalkyl of 1-6 carbon atoms, alkoxyalkyl of 2-8 carbon atoms, carboxy, carboalkoxy of 1-6 carbon atoms, phenyl, carboalkyl of 2-7 carbon atoms, chloro, fluoro, or bromo;
Z is amino, hydroxy, alkoxy of 1-6 carbon atoms, alkylamino wherein the alkyl moiety is of 1-6 carbon atoms, dialkylamino wherein each of the alkyl moieties is of 1-6 carbon atoms, morpholino, piperazino, N-alkylpiperazino wherein the alkyl moiety is of 1-6 carbon atoms, or pyrrolidino;
m=1-4, q=1-3, and p=0-3;
any of the substituents R₁, R₂, R₃, or R₄ that are located on contiguous carbon atoms can together be the divalent radical —O—C(R8)2—O—;
or a pharmaceutically acceptable salt thereof.

4. The method according to claim 1, wherein the EGFR kinase inhibitor is (4-dimethylamino-but-2-enoic acid [4-(3-chloro-4-fluoro-phenylamino)-3-cyano-7-ethoxy-quinolin-6-yl]-amide or a pharmaceutically acceptable salt thereof.

5. The method according to claim 1, wherein the EGFR kinase inhibitor is a compound having the structure: 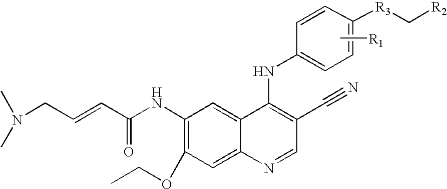
wherein:
R₁ is halogen;
R₂ is a pyridinyl, optionally substituted pyrimidine, thiazole, or an optionally substituted phenyl ring wherein the phenyl or pyrimidine ring may be unsubstituted, mono-substituted, or di-substituted; and
R₃ is —O— or —S—; or a pharmaceutically acceptable salt thereof.

6. The method according to claim 1, wherein the EGFR kinase inhibitor is ((E)-N-{4-[3-chloro-4-(2-pyridinyl methoxy)aniline]-3-cyano-7-ethoxy-6-quinolinyl}-4-(dimethylamino)-2-butenamide or a pharmaceutically acceptable salt thereof.

7. The method according to claim 1, wherein the cancer comprises non-small cell lung cancer.
